(19) United States
(12) Patent Application Publication
(10) Pub. No.: US 2021/0255142 A1
(43) Pub. Date: Aug. 19, 2021

(54) DIAGNOSING APPARATUS, ANALYSIS METHOD, AND PROGRAM

(71) Applicant: NATIONAL INSTITUTE FOR MATERIALS SCIENCE, Tsukuba-shi, Ibaraki (JP)

(72) Inventors: Akihiro OKAMOTO, Tsukuba-shi, Ibaraki (JP); Divya NARADASU, Tsukuba-shi, Ibaraki (JP)

(21) Appl. No.: 17/256,832

(22) PCT Filed: Jul. 5, 2019
(86) PCT No.: PCT/JP2019/026797

§ 371 Date: Dec. 29, 2020

(30) Foreign Application Priority Data
Jul. 9, 2018 (JP) 2018-130027
Dec. 26, 2018 (JP) 2018-242680

(51) Int. Cl. G01N 27/48; G01N 33/487; G01N 27/327

(52) U.S. Cl. CPC G01N 27/48; G01N 27/327; G01N 33/48707

(57) ABSTRACT

[Object] An object of the present technology to provide a diagnosing apparatus capable of providing, more accurately and simply, information for determining at least one item selected from the group consisting of a type of an illness that has developed in a subject, and a state of progress of the illness that has developed in the subject. Another object of the present technology is to provide an analysis method.

[Solving Means] There is provided a diagnosing apparatus including: a cell into which a sample solution including collected body fluid is introduced; at least two electrodes that are disposed in the cell and come into contact with the sample solution; a voltage applying unit that applies a voltage between the electrodes; a measuring unit that measures an electrochemical response when the voltage is applied; a storage unit that stores a reference; and an analysis output unit that compares a numerical value obtained from the response with the reference, and provides information for determining at least one item selected from the group consisting of a type of an illness that has developed in a subject, and a state of progress of the illness that has developed in the subject.

### TECHNICAL FIELD

The present invention relates to a diagnosing apparatus, an analysis method, and a program.

### BACKGROUND ART

Methods for diagnosing a type of an illness that has developed in a subject and/or a state of progress of the illness from body fluid or the like of the subject have been developed.

For example, methods for providing information for diagnosing, using saliva or the like obtained from the oral cavity of a subject, a state of progress of caries and a periodontal disease in the oral cavity are known.

As the technology as described above, Patent Literature 1 describes “a method for performing semi-quantitative measurement of cariogenic bacteria in a plaque sample or a saliva sample, executed by (a) concentrating microbes contained in the plaque sample or the saliva sample as necessary; (b) causing the microbes to come into contact with a carbon source that is fermented to an acid by the cariogenic bacteria; (c) incubating the microbes and the carbon source under conditions that promote selective acid formation by the cariogenic bacteria; and (d) deciding a pH at least once within a period of 12 hours after addition of the carbon source; in which the semi-quantitative measurement of the cariogenic bacteria in the sample is executed by comparing the pH decided in the step (d) with at least one reference value”.

### CITATION LIST

### Patent Literature

Patent Literature 1: Japanese Patent Application Laid-open No. 2012-24085

### DISCLOSURE OF INVENTION

### Technical Problem

The present inventors have found that in the method described in Patent Literature 1, accurate information for determining a type of an illness that has developed in a subject and a state of progress of the illness cannot be provided in some cases. Further, the present inventors have found a problem that the method described in Patent Literature 1 is complicated to operate and time-consuming to provide information.

In this regard, it is an object of the present technology to provide a diagnosing apparatus capable of providing, more accurately and simply, information for determining at least one item selected from the group consisting of a type of an illness that has developed in a subject, and a state of progress of the illness that has developed in the subject. Further, another object of the present technology is to provide an analysis method and a program.

### Solution to Problem

The present inventors have intensively studied to achieve the above-mentioned object, and as a result, have found that the above-mentioned object can be achieved by the following configurations.

[1] A diagnosing apparatus, including: a cell into which a sample solution containing body fluid collected from a subject is introduced; at least two electrodes that are disposed in the cell and come into contact with the sample solution; a voltage applying unit that applies a voltage between the electrodes; a measuring unit that measures an electrochemical response when the voltage is applied; a storage unit that stores a reference that defines a relationship between a numerical value obtained from the response and at least one of a type of an illness or a state of progress of the illness; and an analysis output unit that compares the numerical value obtained from the subject with the reference, and provides information for determining at least one of the type of the illness that has developed in the subject or the state of progress of the illness that has developed in the subject.

[2] The diagnosing apparatus according to [1], in which the numerical value is an absolute value of a current measured by the measuring unit or a difference between the response measured using body fluid obtained from the subject and the response measured using body fluid obtained from at least one of the subject in a healthy state or a healthy person different from the subject.

[3] The diagnosing apparatus according to [1] or [2], in which the body fluid is saliva.

[4] The diagnosing apparatus according to [3], in which the illness is at least one of caries or a periodontal disease.

[5] The diagnosing apparatus according to any one of [1] to [4], in which the electrodes include at least one of a reference electrode or a counter electrode.

[6] The diagnosing apparatus according to any one of [1] to [5], in which the voltage applying unit applies a predetermined voltage between the electrodes.

[7] The diagnosing apparatus according to any one of [1] to [6], in which the voltage applying unit applies a sweep voltage between the electrodes.

[8] The diagnosing apparatus according to any one of [1] to [7], in which the storage unit records the measured response, and the analysis output unit compares the response obtained by measurement of the sample solution with the response that has been obtained by preceding measurement prior to the measurement and recorded in the storage unit, and provides information for determining the at least one of the type of the illness that has developed in the subject or the state of progress of the illness on the basis of the reference from a result of the comparison.

[9] The diagnosing apparatus according to any one of [1] to [8], in which the cell is configured to be hermetically sealable.

[10] The diagnosing apparatus according to any one of [1] to [9], in which a shortest distance between the electrodes in the cell is a distance at which one of cells of a microbe responsible for the illness can be short-circuited.

[11] The diagnosing apparatus according to any one of [1] to [10], in which the shortest distance between the electrodes in the cell is 700 nm to 2,000 nm.

[12] The diagnosing apparatus according to any one of [1] to [11], in which the electrode includes a comb-shaped electrode.

[13] The diagnosing apparatus according to any one of [1] to [12], further including: a transmission unit that transmits the response to an external server via a communication line; and a reception unit that receives at least one of a consultation deadline or expected progress of the illness that has been calculated by machine-learning in an external server on the basis of the response transmitted from the transmission unit.

[14] The diagnosing apparatus according to any one of [1] to [13], further including a reference creating device that create the reference by machine-learning on the basis of the above response.

[15] An analysis method, including: a step of introducing, into a cell in which at least two electrodes are disposed, a sample solution containing body fluid collected from a subject so as to come into contact with the electrodes; a step of measuring an electrochemical response when a voltage is applied between the electrodes; and a step of comparing a reference defining a relationship between a numerical value obtained from the response and at least one of a type of an illness or a state of progress of the illness with the numerical value obtained from the subject to provide information for determining at least one of the type of the illness or the state of progress of the illness in the subject.

[16] The analysis method according to [15], in which the applied voltage is a predetermined voltage.

[17] The analysis method according to [15], in which the applied voltage is a sweep voltage.

[18] The analysis method according to any one of [15] to [17], in which the body fluid is saliva.

[19] The analysis method according to [18], in which the illness is at least one of caries or a periodontal disease.

[20] A program that causes, in a system that includes a diagnosing apparatus and a server that is a computer, the diagnosing apparatus and the server being configured to communicate with each other via a network, the diagnosing apparatus including a cell into which a sample solution containing body fluid collected from a subject is introduced, at least two electrodes that are disposed in the cell and come into contact with the sample solution, a voltage applying unit that applies a voltage between the electrodes, a measuring unit that measures an electrochemical response when the voltage is applied, a storage unit that stores a reference that defines a relationship between a numerical value obtained from the response and at least one of a type of an illness or a state of progress of the illness, an analysis output unit that compares the numerical value obtained from the subject with the reference, and provides information for determining at least one of the type of the illness that has developed in the subject or the state of progress of the illness that has developed in the subject, and a display unit, the computer to execute the procedures of; receiving a specific response determined to be abnormal by comparing the numerical value and the reference, of the response obtained by the diagnosing apparatus; applying the specific response to an estimation model to estimate generation of pathogenic microbes in the sample solution, the estimation model being learned to estimate, when an electrochemical response is input, presence or absence of generation of the pathogenic microbes responsible for the illness, learning having been performed in advance in the estimation model by learning data including an electrochemical response of a learning sample solution different from the sample solution and a microbiota analysis result of the learning sample solution; and prompting, where generation of the pathogenic microbes is expected, the display unit to display that microbiota analysis is necessary.

[21] A program that causes, in a system that includes a diagnosing apparatus, a server that is a computer, and an analysis device capable of performing microbiota analysis, the diagnosing apparatus, the server, and the analysis device being configured to communicate with each other via a network, the diagnosing apparatus including a cell into which a sample solution containing body fluid collected from a subject is introduced, at least two electrodes that are disposed in the cell and come into contact with the sample solution, a voltage applying unit that applies a voltage between the electrodes, a measuring unit that measures an electrochemical response when the voltage is applied, a storage unit that stores a reference that defines a relationship between a numerical value obtained from the response and at least one of a type of an illness or a state of progress of the illness, an analysis output unit that compares the numerical value obtained from the subject with the reference, and provides information for determining at least one of the type of the illness that has developed in the subject or the state of progress of the illness that has developed in the subject, and a display unit, the computer to execute the procedures of; receiving a specific response determined to be abnormal by comparing the numerical value and the reference, of the response obtained by the diagnosing apparatus, and a sample ID corresponding to the sample solution; applying the specific response to an estimation model to estimate generation of pathogenic microbes in the sample solution, the estimation model being learned to estimate, when an electrochemical response is input, presence or absence of generation of the pathogenic microbes responsible for the illness, learning having been performed in advance in the estimation model by learning data including an electrochemical response of a learning sample solution different from the sample solution and a microbiota analysis result of the learning sample solution; prompting, where generation of the pathogenic microbes is expected, the display unit to display that microbiota analysis is necessary for the sample solution; receiving a result of microbiota analysis of the measurement solution and the sample ID from the analysis device; deciding presence or absence of pathogenic microbes in the sample solution from the result of microbiota analysis; and prompting, where it is decided from the result of microbiota analysis that the pathogenic microbes are not generated, the diagnosing apparatus to change the reference.

### Advantageous Effects of Invention

In accordance with the present invention, it is possible to provide a diagnosing apparatus capable of providing, more accurately and simply, information for determining at least one item selected from the group consisting of a type of an illness that has developed in a subject, and a state of progress of the illness that has developed in the subject. Further, in accordance with the present invention, it is possible to provide an analysis method and a program.

### BRIEF DESCRIPTION OF DRAWINGS

FIG. 1 is a schematic diagram showing a diagnosing apparatus according to a first embodiment of the present invention.

FIG. 2 is a transmission electron microscopy image of a section stained under acidic conditions (pH4), i.e., to be redox reaction-specific to cultured Streptococcus mutans bacteria.

FIG. 3 is a transmission electron microscopy image of a section stained to be redox reaction-specific to Streptococcus mutans bacteria cultured under neutral conditions.

FIG. 4 is a diagram showing a temporal change in the glucose oxidation current in the case where a solution containing bacteria and glucose is measured using a diagnosing apparatus according to an embodiment of the present invention.

FIG. 5 is an electron micrograph of Capnocytophaga ochracea bacteria cultured under anaerobic conditions.

FIG. 6 is a diagram showing a temporal change in the current of Capnocytophaga ochracea bacteria cultured under anaerobic conditions.

FIG. 7 is an explanation diagram for describing an example of a method of performing comparison by an analysis output unit.

FIG. 8 is a schematic graph showing the magnitude of the current value with respect to the potential difference between electrodes of a sample solution D3 and a sample solution D4 in the case where a sweep voltage is applied between the electrodes.

FIG. 9 shows an example of an analytical flow using the diagnosing apparatus according to the embodiment of the present invention.

FIG. 10 shows an example of another analytical flow using the diagnosing apparatus according to the embodiment of the present invention.

FIG. 11 is a perspective view showing a biosensor of a diagnosing apparatus according to a second embodiment of the present invention.

FIG. 12 is a perspective view showing a state in which an upper insulating substrate of a biosensor is separated.

FIG. 13 is a top view of the diagnosing apparatus according to the embodiment of the present invention.

FIG. 14 is a diagram showing an example of display content in the case where the current value in the sample solution is greater than or equal to a reference.

FIG. 15 is a diagram showing an example of display content in the case where the current value in the sample solution is greater than or equal to the reference.

FIG. 16 is a diagram showing an example of display content in the case where the current value in the sample solution is greater than or equal to the reference.

FIG. 17 is a diagram describing a functional configuration of a diagnosing apparatus including a measuring device and a biosensor.

FIG. 18 is a top view showing another embodiment of a biosensor that can be used in the diagnosing apparatus according to the embodiment of the present invention.

FIG. 19 is a top view schematically showing a state in which the upper insulating substrate of the biosensor has been removed.

FIG. 20 is a schematic diagram showing another embodiment of a comb-shaped electrode.

FIG. 21 is a schematic diagram showing another embodiment of the comb-shaped electrode.

FIG. 22 is a schematic diagram showing a diagnosing apparatus according to a third embodiment of the present invention.

FIG. 23 is a functional explanatory diagram of a diagnosing apparatus including a measuring device, a biosensor, and a reference creating device that creates a reference by machine-learning on the basis of a current value.

FIG. 24 is a schematic diagram showing a system including a diagnosing apparatus.

FIG. 25 is a schematic diagram of a system that includes a diagnosing apparatus and a server that is a computer, the diagnosing apparatus and the server being configured to be capable of communicating with each other via a network.

FIG. 26 is a process flow of a program according to a first embodiment of the present invention.

FIG. 27 shows an example of displaying that microbiota analysis is necessary, which is displayed on a diagnosing apparatus.

FIG. 28 is a schematic diagram showing a system that includes a diagnosing apparatus, a server that is a computer, and an analysis device capable of performing microbiota analysis, the diagnosing apparatus, the server, and the analysis device being configured to be capable of communicating with each other via a network.

FIG. 29 is a process flow of a program according to a second embodiment of the present invention.

### MODE(S) FOR CARRYING OUT THE INVENTION

Hereinafter, the present invention will be described in detail.

Description of the components set forth below is made on the basis of typical embodiments of the present invention in some cases, but the present invention is not limited to such embodiments.

Note that in the present specification, a numerical value range represented by “to” means a range including the numerical values described before and after “to” as the lower limit value and the upper limit value.

Further, in the present specification, a state of progress of the illness means at least one item selected from the group consisting of presence or absence of the illness, the risk of the illness (risk of morbidity), and degree of progress of the illness.

### First Embodiment of Diagnosing Apparatus

A diagnosing apparatus according to an embodiment of the present invention is a diagnosing apparatus including: a cell into which a sample solution containing body fluid collected from a subject is introduced; at least two electrodes that are disposed in the cell and come into contact with the sample solution; a voltage applying unit that applies a voltage between the electrodes; a measuring unit that measures an electrochemical response when the voltage is applied; a storage unit that stores a reference that defines a relationship between a numerical value obtained from the response and at least one item selected from the group consisting of a type of an illness and a state of progress of the illness; and an analysis output unit that compares the numerical value obtained from the subject with the reference, and provides information for determining at least one item selected from the group consisting of the type of the illness that has developed in the subject, and the state of progress of the illness that has developed in the subject.

In the following, first, as one embodiment of the present invention, cases where the body fluid is saliva and the illness is caries and a periodontal disease will be described.

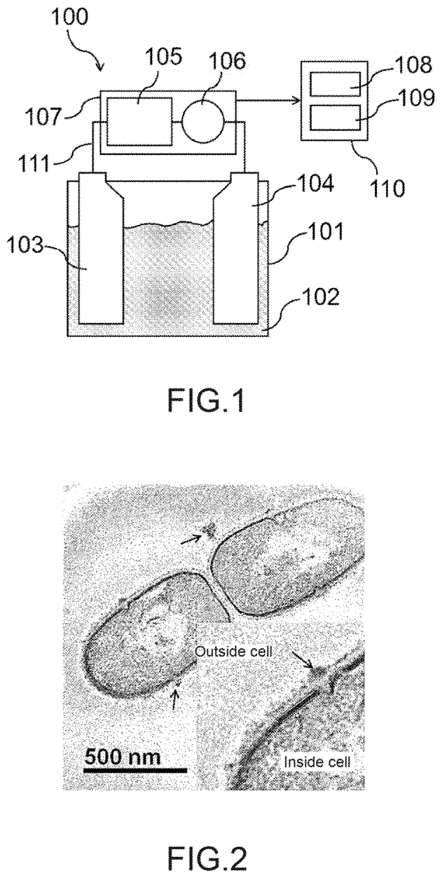

FIG. 1 is a schematic diagram showing a diagnosing apparatus according to a first embodiment of the present invention. A diagnosing apparatus 100 includes a cell 101 for introducing a sample solution 102 containing saliva obtained from the oral cavity of a subject, and a first electrode 103 and a second electrode 104 are disposed in the cell 101 so as to come into contact with the introduced sample solution 102.

The first electrode 103 and the second electrode 104 are electrically connected to each other via a circuit 111, and a voltage applying unit 105 for applying a voltage between the first electrode 103 and the second electrode 104 is disposed on the circuit 111. Further, a measuring unit 106 for measuring an electrical response when a voltage is applied between the first electrode 103 and the second electrode 104 by the voltage applying unit 105, which is typically a current value flowing between the electrodes (the circuit 111), is disposed in the same manner.

In the diagnosing apparatus 100, the voltage applying unit 105 and the measuring unit 106 constitute a potentiostat 107.

Note that although the voltage applying unit 105 and the measuring unit 106 in the diagnosing apparatus 100 constitute the potentiostat 107, the diagnosing apparatus according to the embodiment of the present invention is not limited to the above, and the voltage applying unit 105 and the measuring unit 106 may be independently provided.

The diagnosing apparatus 100 includes a terminal 110 connected to the potentiostat 107 so as to be capable of performing data communication with each other, and the terminal 110 includes a storage unit 108 and an analysis output unit 109.

Note that the terminal 110 further includes a control unit (not shown) that is a processor, and the control unit controls the storage unit 108 and the analysis output unit 109. Further, the control unit may control the potentiostat 107.

Note that the terminal 110 is typically a computer including a display device (display unit). As the processor of the control unit, a processor such as a CPU (central processing unit) and a GPU (graphics processing unit) can be used. The control unit reads the program stored in the storage unit and performs predetermined arithmetic processing in accordance with the program.

Further, the control unit is capable of writing the operation result obtained by the analysis output unit to the storage unit as appropriate and reading it from the storage unit.

Further, the storage function of the storage unit can be realized by a non-volatile memory such as an HDD (hard disk drive) and an SSD (solid state drive). Further, the storage unit may have a function as a memory for writing or reading intermediate progress or the like of the arithmetic processing by the analysis output unit. The memory function of the storage unit can be realized by a volatile memory such as a RAM (Random Access memory) and a DRAM (Dynamic Random Access memory).

Using the above-mentioned diagnosing apparatus 100, the procedures for providing information for determining a state of progress of an illness in the oral cavity of a subject and the principles of measurement are described below.

In the past, the acidification of biofilms responsible for caries has been thought to be caused by lactic acid generated by bacteria by degrading sugars. However, the present inventors have found for the first time that the acidification of the biofilm progresses as electric bacteria move electrons to electrodes.

Further, the present inventors have found that Streptococcus mutans and Streptococcus sobrinus, which are typical caries-causing bacteria, and the like express electrochemical activity specifically under acidic conditions and perform current generation.

The present invention is completed on the basis of the new finding that “pathogenic” caries-causing bacteria can be specifically and electrochemically detected.

FIG. 2 shows a transmission electron microscopy image of a section stained under acidic conditions (pH4), i.e., to be redox reaction-specific to cultured Streptococcus mutans bacteria. The surface of the cell wall and the inner cell membrane are stained, suggesting that an enzyme having electron transfer ability is expressed.

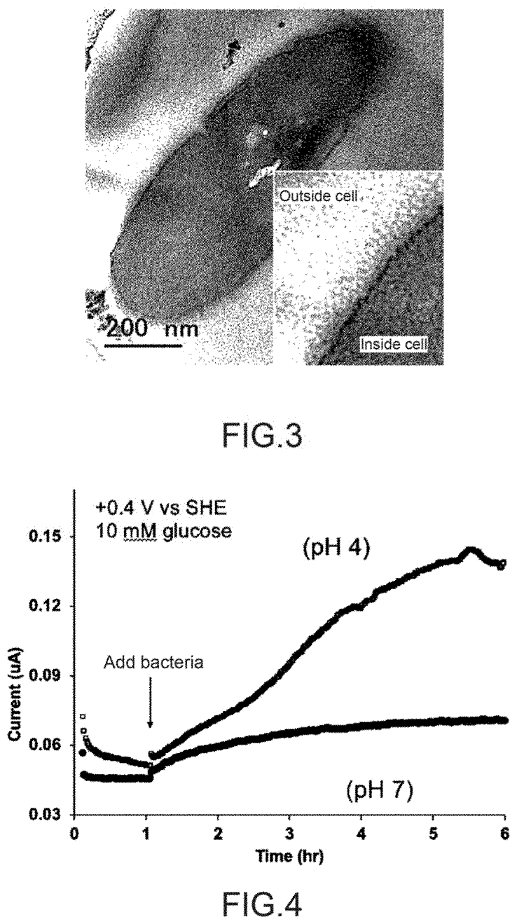

FIG. 3 shows a transmission electron microscopy image of a section stained to be redox reaction-specific to Streptococcus mutans bacteria cultured under neutral conditions. Although the cell membrane inside the cell wall is stained, the contrast with the inside of the cell is weak as compared with FIG. 2, and the surface of the cell wall is not stained, which suggests that the enzyme having the electron transfer ability is not expressed under the neutral culture conditions as compared with the acidic culture conditions.

Next, FIG. 4 shows a temporal change (i.e., electrochemical response when a voltage is applied) in the glucose oxidation current in the case where a solution containing the bacteria and glucose is measured using the diagnosing apparatus according to the embodiment of the present invention. Note that an electrode formed of indium tin oxide was used for the electrode.

In accordance with FIG. 4, it was shown that the current value was raised specifically (line described as “pH4” in FIG. 4) only under acidic conditions, i.e., when the bacteria are in the state that exerts the pathogenicity (hereinafter, the microbe in such a state will be referred to also as “pathogenic microbes” in the present specification). This is presumably because as shown in FIG. 2 and FIG. 3, electron-transfer enzymes are expressed specifically in the extracellular membrane of Streptococcus mutans bacteria under acidic conditions.

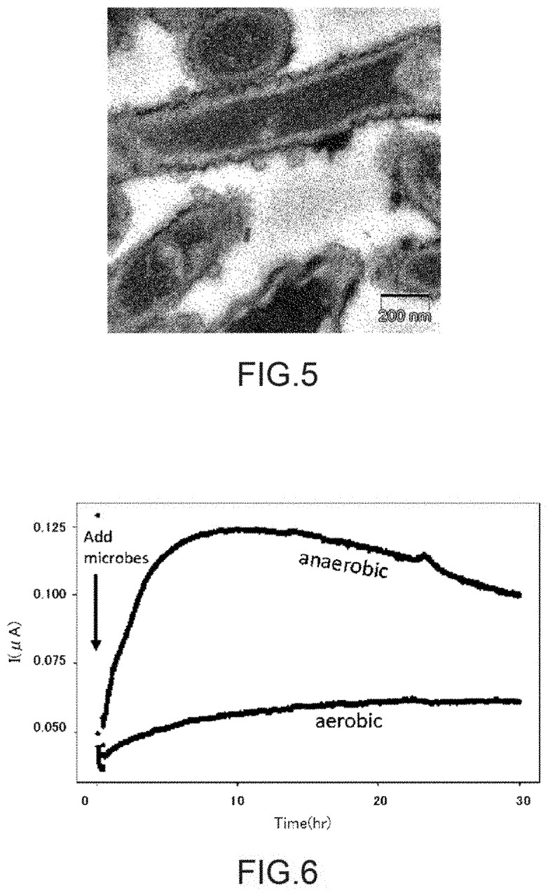

The present inventors have confirmed that this analysis method can be applied to microbes responsible for an illness in the oral cavity other than caries-causing bacteria. FIG. 5 is an electron micrograph of Capnocytophaga ochracea bacteria cultured under anaerobic conditions. Although Capnocytophaga ochracea bacteria are each a type of so-called opportunistic bacterium, it is known to be responsible for a periodontal disease.

The expression of electron-transfer enzymes has been confirmed by culturing Capnocytophaga ochracea bacteria under anaerobic conditions in which pathogenicity is exerted.

Further, at this time, by measuring a response using a diagnosing apparatus similar to that described above (FIG. 6), it has been found that electrochemical properties are specifically expressed under anaerobic conditions and the presence of pathogenic bacteria can be easily detected.

The present inventors completed the present invention with the idea of being capable of accurately detecting the presence of “pathogenic (having pathogenicity)” microbes (typically bacteria) contained in the sample solution by measuring the above-mentioned response. That is, the present invention has been completed with the idea that whether or not pathogenic microbes, of microbes responsible for an illness in the oral cavity, which are each a type of a so-called opportunistic bacterium, are present in the sample solution can be detected by measuring an electrochemical response.

The existing caries detection method and diagnosing apparatus (Patent Literature 1) have been used to detect the above-mentioned caries-causing microbes themselves and/or the metabolites thereof, or the like. However, the microbes responsible for caries are known as so-called opportunistic bacteria, and presumed to be present also in the oral cavity of a healthy person. The existing caries detection method does not take into account at all whether or not the bacteria are present in the oral cavity of a subject in a caries-causing (pathogenic) state. The present inventors have presumed that this is a cause of the obtained results being inaccurate.

In the diagnosing apparatus according to the embodiment of the present invention, electric characteristics newly found by the present inventors that microbes according to an illness in the oral cavity express electron-transfer enzymes specifically in the pathogenic state and electric current generation is observed accordingly as described above are used as measuring principles. Therefore, information for determining a state of progress of an illness in the oral cavity of a subject can be provided more accurately and easily.

Note that the cases where body fluid is saliva and the illness is caries and a periodontal disease have been described above, but the measuring device according to the embodiment of the present invention is not limited to the above description and can be applied to the illness caused by microbes (particularly bacteria) regardless of the type of the body fluid and the type of the illness.

The body fluid applicable to the diagnosing apparatus according to the embodiment of the present invention is not particularly limited. Examples thereof include blood, lymph fluid, tissue fluid, body cavity fluid, digestive fluid, sweat, tears, runny nose, urine, semen, vaginal fluid, amniotic fluid, and milk.

Further, examples of the illness include, but particularly not limited to, a eubacterial infection and a fungal infection.

### <Sample Solution>

The sample solution applicable to the diagnosing apparatus according to the embodiment of the present invention is not particularly limited as long as it contains body fluid of a subject. The body fluid is as described above.

The method of obtaining the sample solution is not particularly limited. Examples of the method include, but not particularly limited to, directly collecting body fluid of a subject, collecting body fluid from a member wiping off the body of a subject, and washing the body of a subject with a buffer to collect the cleaning liquid.

Among them, saliva is favorable as the body fluid. The saliva contains bacteria in the oral cavity of a subject. In accordance with the diagnosing apparatus according to this embodiment, it is possible to provide information for determining the presence or absence of bacteria in a pathogenic state and the growth state of the bacteria in the oral cavity of a subject. As a result, it is possible to provide information for determining the type of an illness that has developed in the oral cavity and a state of progress of the illness.

In the case where the sample solution contains saliva, the subject is not particularly limited. As the subject, an animal having a tooth (particularly a mammal) is favorable, and a human is favorable. Examples of mammals other than a human include, but not limited to, dogs, cats, horses, cows, pigs, sheep, goats, donkeys, mules, camels, llamas, alpacas, tortoises, zebus, yaks, guinea pigs, rabbits, foxes, fennec foxes, and monkeys.

The sample solution only needs to contain saliva and may contain components other than saliva. Examples of the components other than saliva include a solvent (water and/or an organic solvent) and an additive. The additive is not particularly limited. Examples of the additive include organic substances (e.g., glucose, yeast extract, etc.), which are utilized in microbes.

The sample solution 102 is introduced into the cell 101 so as to come into contact with the first electrode 103 and the second electrode 104 in the diagnosing apparatus 100. The method of introducing the sample solution 102 into the cell 101 is not particularly limited. The sample solution may be introduced directly from the oral cavity of a subject, or a sample solution prepared by adding a solvent and/or an additive to saliva obtained from the oral cavity of a subject as necessary may be introduced.

The voltage applying unit 105 is capable of controlling a voltage value applied between the first electrode 103 and the second electrode 104 (referred to also as “between electrodes”), timing, and the like.

In the diagnosing apparatus 100, the above-mentioned voltage applying unit 105 constitutes the potentiostat 107, but the diagnosing apparatus according to the embodiment of the present invention is not particularly limited. The voltage applying unit 105 only needs to be capable of applying a desired voltage between the first electrode 103 and the second electrode 104.

The operator (who may be an operator other than a subject, or the subject himself/herself) of the diagnosing apparatus operates the potentiostat 107 to apply a voltage between the electrodes by the voltage applying unit 105. At this time, the voltage applied between the electrodes may be constant (a constant voltage), or may be increased or decreased in accordance with the time of application. In the case of increasing or decreasing the voltage to be applied, the voltage may be changed in a stepwise manner in accordance with the time of voltage application, or the voltage may be changed in a continuous manner in accordance with the time of voltage application. That is, the voltage to be applied may have a predetermined value (constant value), may be a sweep voltage, or may be a combination thereof.

In the diagnosing apparatus 100, an operator operates the potentiostat 107 to apply a voltage between the electrodes by the voltage applying unit 105, but the diagnosing apparatus according to the embodiment of the present invention is not limited to the above. For example, a potentiostat (as a result, a voltage applying unit) may be controlled from a control unit of the terminal 110 connected to the potentiostat so as to be capable of performing data communication with each other. In this case, the operator is capable of applying a voltage between the electrodes by operating a terminal.

Further, as described below (e.g., a diagnosing apparatus according to a second embodiment), in the case of a diagnosing apparatus where a voltage applying unit and a control unit are integrally formed, a voltage may be applied by operating the diagnosing apparatus itself.

When a voltage is applied between the electrodes, in the case where pathogenic microbes (typically pathogenic bacteria) are present in the sample solution, a metabolic current due to oxidation of organic substances and/or an oxidation-reduction current due to electron-transfer enzymes expressed in the extracellular membrane are generated, and the above-mentioned response is detected in the measuring unit.

It is generally known that saliva contains 10⁸ to 10¹¹ CFU/ml of 100 types or more of bacteria. In these bacteria, bacteria responsible for an illness and bacteria that do not cause an illness are mixed, and pathogenic and non-pathogenic bacteria are mixed even in such bacteria responsible for an illness.

In accordance with a diagnosing apparatus according to an embodiment of the present invention, in the case where bacteria responsible for an illness in the oral cavity are present in a pathogenic state among the above-mentioned bacteria, it is possible to provide information for specifically detecting the above-mentioned current value and determining a state of progress of the illness in the oral cavity of a subject.

### <Electrode>

There is no particular limitation on the material of the electrode, and a known electrode material can be used. Examples of the electrode material include carbon, gold, platinum, silver, molybdenum, cobalt, nickel, palladium, and ruthenium. Indium tin oxide or the like may be used, and a known material for an electrode can be used.

Further, there is no particular limitation on the shapes and the like, and a known electrode for an electrochemical measurement cell can be used. Among them, it is favorable that the area of the electrode in contact with the sample solution is 1 cm² or less because even a smaller sample solution (specifically, 0.1 to 5 ml) can be detected sensitively.

### <Cell>

The cell is not particularly limited, and a cell known for electrochemical measurement can be used. The cell is favorably made of an insulating material. For example, the cell is formed of an insulating material, e.g., a thermoplastic resin such as polyetherimide (PEI), polyethylene terephthalate (PET), and polyethylene (PE), a thermosetting resin such as a polyimide resin and an epoxy resin, glass, ceramic, or paper. Although there is no particular limitation on the size of the cell, the size can be appropriately selected in accordance with the amount of the sample solution to be used, and is favorably a volume of approximately 0.1 to 5 ml.

Further, the cell may be configured to be hermetically sealable. In the case where the cell is hermetically configured, more sensible measurement can be performed in some cases. There is no particular limitation on the method of hermetically configuring the cell, and a known method can be applied. Examples thereof include a method of using a cell with a lid, which includes a cell and a lid portion covering an opening of the cell.

The measured response is compared with a reference in the analysis output unit 109 by using a numerical value obtained from the response. As a result, information for determining at least one item selected from the group consisting of the type of an illness that has developed in the oral cavity of a subject and a state of progress of the illness is provided (output to the outside) from the analysis output unit 109.

Here, the reference is a reference defining a relationship between a numerical value obtained from an electrochemical response and at least one item selected from the group consisting of the type of the illness that has developed and a state of progress of the illness, and is stored in the storage unit 108 in advance.

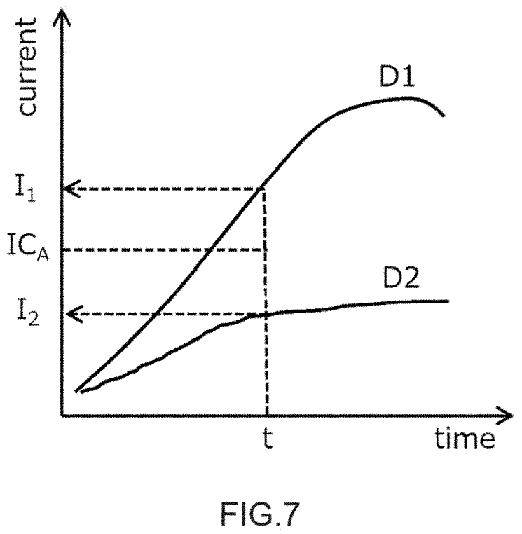

FIG. 7 is an explanatory diagram for describing an example of the above-mentioned method of performing comparison by the analysis output unit. FIG. 7 is a schematic graph showing the relationship (electric response) between current values obtained from a sample solution D1 and a sample solution D2 and the time of voltage application (measurement time) in the case where a predetermined voltage is applied between the electrodes (constant potential measurement).

The reference in FIG. 7 is the magnitude of the current value at a predetermined measurement time, and is stored in the storage unit 108 as a predetermined value IC_{A} by machine-learning or the like in relation to the presence or absence of the development of an illness (typically caries) in the oral cavity of a subject.

Here, in the case where the current value detected at a time t is I₁ when the sample solution D1 is measured, the analysis output unit compares this value with IC_{A} and outputs an indication that there is a possibility that an illness (typically caries) has developed in the oral cavity of the subject.

Note that the output content may be, for example, the value itself of the difference between I₁ and IC_{A}, or both the value of the difference and information relating to the possibility of the development of an illness. Further, I₁ and/or the value of the difference between I₁ and IC_{A} can be used also as diagnostic markers for an illness.

Meanwhile, in the case where the current value detected at the time t is I₂ when the sample solution D2 is measured, the analysis output unit compares this value with IC_{A} and outputs an indication that there is no possibility of an illness that has developed in the oral cavity of the subject. The output content in this case is similar to that described above.

Note that although the reference in FIG. 7 is the magnitude of the current value at a predetermined measurement time, the reference in the diagnosing apparatus according to the embodiment of the present invention is not limited to the above, and only needs to be a numerical value obtained from the measured response. As such a numerical value, for example, the absolute value of the current, the rate of change of the current (dI/dt, first order differential value), the second order differential value (d²I/dt²), the inflection point of dI/dt (time until the rise of the current value), the maximum value of the current, the time until the maximum value of the current is obtained, or the like may be used.

The numerical value to be compared with the reference is favorably the absolute value of the current or the difference (e.g., the difference in the shape of the graph of time-current values) from the response measured using body fluid (typically saliva) obtained from the oral cavity of at least one selected from the group consisting of a subject in a healthy state and a healthy person different from the subject.

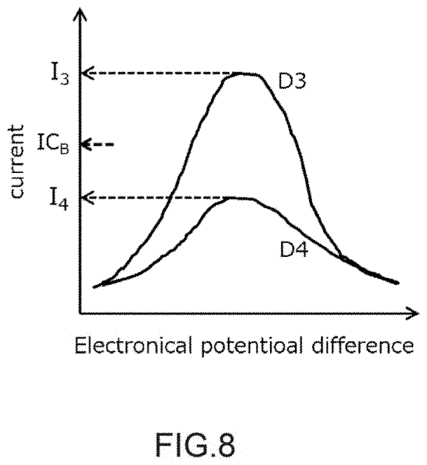

Further, as another embodiment, a schematic graph showing the magnitude of the current value with respect to the potential difference between electrodes of a sample solution D3 and a sample solution D4 in the case where a sweep voltage is applied between the electrodes is shown in FIG. 8.

Here, in the case where the maximum value of the current detected for the sweep voltage is I₃ when the sample solution D3 is measured, the analysis output unit compares this value with a reference IC_{B} and outputs an indication that there is a possibility that an illness (typically caries) has developed in the oral cavity of the subject.

Note that the output content may be, for example, the value itself of the difference between I₃ and IC_{B}, or may be both the value of the difference and information regarding the possibility of the development of an illness. I₃ and/or the difference between I₃ and IC_{B} can be used as diagnostic markers for an illness.

Meanwhile, in the case where the maximum value of the current value detected for the sweep voltage is I₄ when the sample solution D4 is measured, the analysis output unit compares this value with IC_{B} and outputs an indication that there is no possibility of an illness that has developed in the oral cavity of the subject. The output content in this case is similar to that described above.

Note that the reference in FIG. 8 is the maximum value of the current obtained for the sweep voltage, but the reference in the diagnosing apparatus according to the embodiment of the present invention is not limited to the above, and may be, for example, the number of peaks and/or the widths of the peaks.

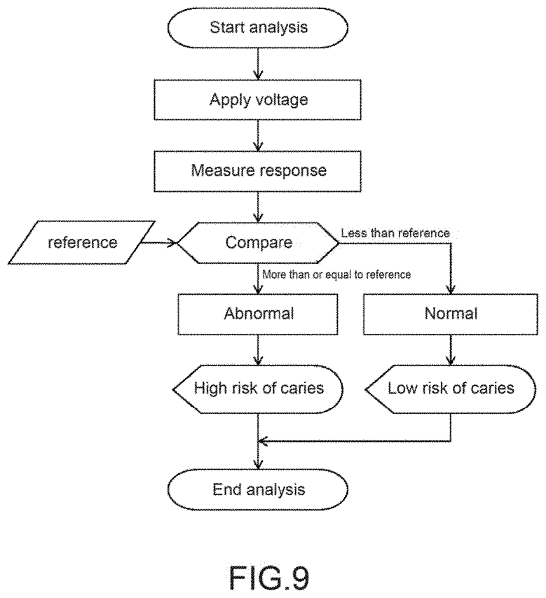

FIG. 9 shows an example of an analytical flow using the diagnosing apparatus according to the embodiment of the present invention. First, a sample containing saliva is introduced into a cell and the analysis is started. Next, a voltage is applied between the electrodes and the resulting response (here, the current value) is measured. The resulting current value is compared with the reference, the current value is treated as an abnormal value in the case where the current value is greater than or equal to the reference, and “high risk of caries” indicating that there is a high possibility of the development of an illness in the oral cavity of a subject is output.

Meanwhile, the current value is treated as a normal value in the case where the current value is lower than the reference, “low risk of caries” indicating that the risk of an illness in the oral cavity of a subject is low is output, and the analysis is completed.

Note that “high risk of caries” and “low risk of caries” are described as output content in FIG. 9, but the diagnosing apparatus according to the embodiment of the present invention is not particularly limited. Indications that caries are absent (or has not developed) in the case of a normal value and caries are present (or has developed) in the case of an abnormal value may be shown by output other than the above. Further, the obtained current value itself and/or the relationship between the reference and the measured value (typically, the difference between the reference and the measured value) may be shown.

Further, the value greater than or equal to the reference is treated as the abnormal value when performing comparison with the reference value in the above-mentioned embodiment, but the value exceeding the reference may be diagnosed as the abnormal value. In such a case, it is favorable to diagnose the value lower than or equal to the reference as the normal reference.

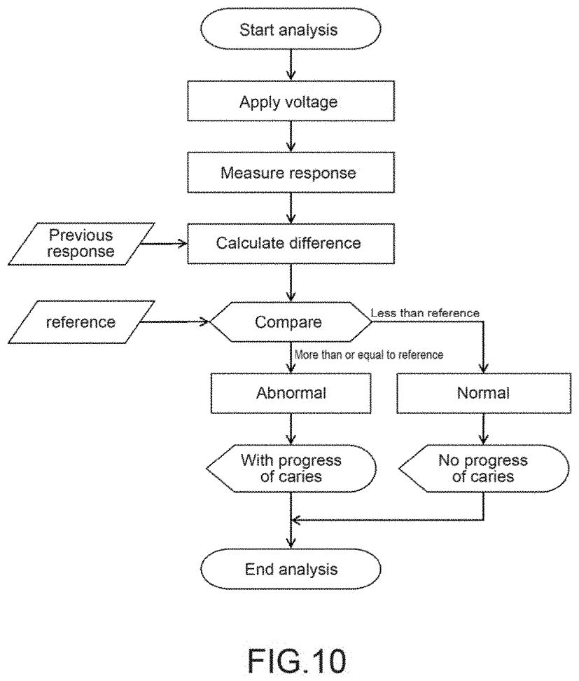

FIG. 10 shows an example of another analytical flow using the diagnosing apparatus according to the embodiment of the present invention. First, a sample containing saliva is introduced into a cell and diagnosis is started. Next, a voltage is applied between the electrodes and the resulting response (here, the current value) is measured. Next, the difference between the obtained current value and the previous measurement value recorded in the storage unit is calculated.

It is favorable that the previous measurement value is a measurement value of a different sample solution, which contains saliva obtained at another time from the same subject. For example, it is favorable that the value is a value measured for a sample solution containing saliva obtained from the same subject at substantially the same time of the previous day.

Next, the resulting difference is compared with the reference, and is treated as an abnormal value in the case where it is greater than or equal to the reference.

In this case, the difference between the current value (previous current value) measured previously for another sample solution containing saliva obtained from the same subject and the current value obtained this time is calculated, and can be compared with the reference to provide information for determining the presence or absence of progress of an illness (typically caries) in the oral cavity.

That is, in the case where the difference between the current values and the reference is equal to or greater than the reference, there is a possibility that an illness (typically caries) has progressed, and “With progress of caries” is output. Meanwhile, in the case where the difference is lower than the reference, the value is treated as a normal value, it is highly likely that caries have not progressed in the oral cavity in the subject, and therefore, “no progress of caries” is output. Thus, the analysis is completed.

Note that “with progress of caries” and “no progress of caries” are described as the output content in FIG. 10, but the output content according to the diagnosing apparatus according to the embodiment of the present invention is not particularly limited. Indications that caries have not progressed in the case of a normal value and caries have progressed in the case of an abnormal value may be shown by output other than the above.

Further, in the above-mentioned embodiment, for example, in the case where the current value under a particular electrode condition is compared with the reference, the value greater than or equal to the reference is treated as an abnormal value, but the value exceeding the reference may be diagnosed as an abnormal value. In this case, it is favorable to diagnose the value lower than or equal to the reference is diagnosed as a normal value.

Conventionally, it is determined when a dentist visually recognizes a black spot in the enamel of a target tooth of a subject (patient) that caries have been generated, and treatment of removing the part where caries have been generated, or the like has been performed.

In accordance with the diagnosing apparatus according to the embodiment of the present invention, since the possibility of generation of caries can be detected at a stage before a black spot is observed, the advantage that the subsequent treatment can be less severe is provided.

### Second Embodiment of Diagnosing Apparatus

A diagnosing apparatus according to a second embodiment of the present invention is a diagnosing apparatus including: a cartridge type biosensor including a first electrode, a second electrode, and a cell; and a measuring device including a voltage applying unit, a measuring unit, a storage unit, an analysis output unit, and a control unit that controls the respective units described above, in which the electrodes of the biosensor are electrically connectable to the voltage applying unit and the measuring unit of the measuring device.

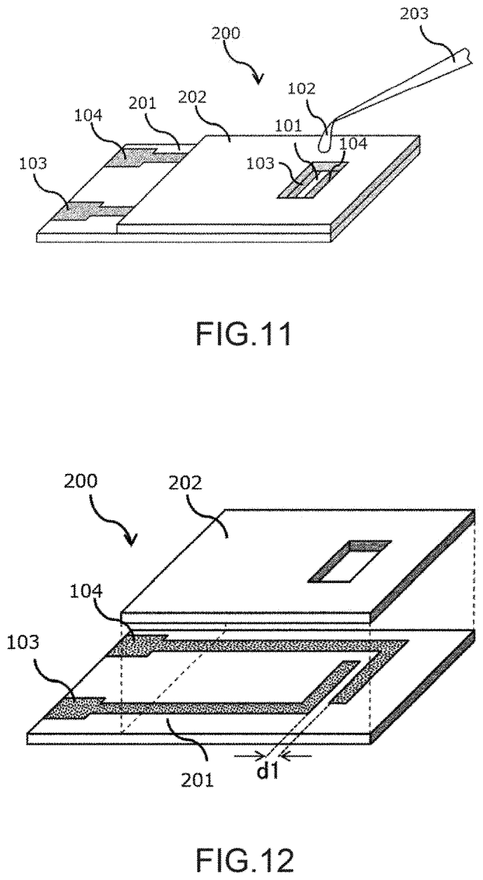

FIG. 11 is a perspective view showing a biosensor of a diagnosing apparatus according to a second embodiment of the present invention.

In a biosensor 200 in FIG. 11, a film-like first electrode 103 and a film-like second electrode 104 are disposed on a lower insulating substrate 201.

The biosensor 200 includes the cell 101 formed by an upper insulating substrate 202 and the lower insulating substrate 201, and the sample solution 102 can be added thereto dropwise by a pipette 203.

Note that the sample solution may be added dropwise by those other than the pipette.

FIG. 12 is a perspective view showing a state in which the upper insulating substrate 202 of the biosensor 200 is separated. The first electrode 103 and the second electrode 104 in the biosensor 200 can be formed using, for example, a known photolithography technology or a known printing technology.

The shortest distance between the first electrode 103 and the second electrode 104 in the biosensor 200, i.e., a distance d1 between the two electrodes in the part where the first electrode 103 and the second electrode 104 are closest to each other, is not particularly limited, but is favorably 10 to 3,000 nm in terms of achieving a diagnosing apparatus having a faster response rate.

When the shortest distance dl between the first electrode 103 and the second electrode 104 is within the above-mentioned range, a current flows between the electrodes having a potential difference when bacteria responsible for an illness in the oral cavity, which are in a pathogenic state, bridge the electrodes. That is, when bacteria in the pathogenic state are present in the sample solution, the bacteria can be detected immediately by detecting the above-mentioned current value.

Specifically, it is presumed that a current value of approximately 100 pA can be measured when one of the above-mentioned bacteria bridges the electrodes having a potential difference of 50 mV.

In accordance with the diagnosing apparatus according to the above-mentioned embodiment, it is possible to achieve an effect that the time from introducing a sample solution into a cell to detecting the current becomes shorter.

The shortest distance d1 can be appropriately selected in accordance with the size of the bacterium to be detected. Above all, in the case where the distance d1 is a distance at which one of the cells of the microbe (typically the bacterium) responsible for an illness can be short-circuited, it is favorable because information for determining a type of an illness that has developed in a subject in the above-mentioned diagnosing apparatus can be easily and quickly provided.

For example, a case where the distance d1 is 700 nm to 2,000 nm will be described.

Assumption is made that a sample solution containing saliva contains bacteria responsible for caries (e.g., Streptococcus mutans) and bacteria responsible for a periodontal disease (e.g., Porphyromonas gingivalis (hereinafter, PG bacteria) or Aggregatibacter actinomycetem comitans (hereinafter, AA bacteria)).

At this time, the bacteria responsible for caries are Streptococcus sp. and the size of one cell thereof is approximately 400 to 500 nm in diameter. Meanwhile, PG bacteria and AA bacteria are anaerobic bacilli and facultative anaerobic bacilli, and the size of one cell thereof is approximately 1 to 2 μm.

At this time, when the distance d1 is 700 nm to 2,000 nm, in the case where bacteria responsible for a periodontal disease are present in a sample solution, the above-mentioned bacteria adhere between the electrodes, and the electron-transfer enzymes expressed in the extracellular membrane cause the electrodes to be short-circuited, which can be selectively detected. Meanwhile, the size of each of the caries-causing bacteria is less than the distance dl between the electrodes, and therefore, they are not detected instantaneously.

As described above, by setting the distance dl in accordance with the size of each of the cells of the bacteria to be measured, the bacteria to be measured can be selectively, easily, and quickly measured.

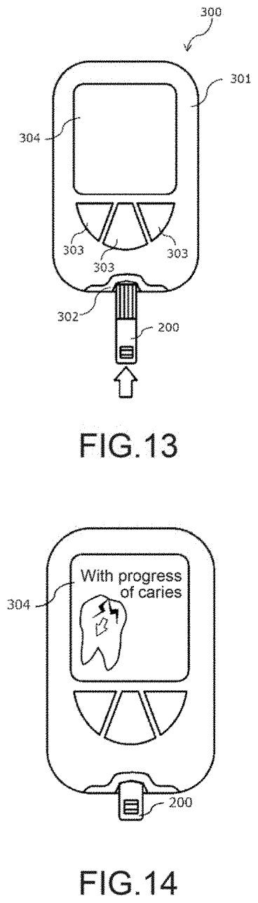

FIG. 13 is a top view of a diagnosing apparatus 300 according to this embodiment. The diagnosing apparatus 300 includes the biosensor 200 described above and a measuring device 301, and is used by inserting the biosensor 200 into a sensor insertion hole 302 of the measuring device 301. When the biosensor 200 is inserted into the sensor insertion hole 302, a measuring unit and a voltage applying unit (not shown) in the measuring device 301 are electrically connected to electrodes of the biosensor 200, and the measuring device 301 can be used.

In accordance with the diagnosing apparatus according to this embodiment, the biosensor 200 can be replaced for each sample solution, and analysis can be performed in a state in which the inside of the cell including the electrode surface is clean. This makes it possible to provide more accurate results.

The measuring device 301 includes an operation button 303 and a display unit 304 on a casing. The operator of the diagnosing apparatus 300 is capable of performing measurement by adding a sample solution to a cell of the biosensor 200 dropwise, inserting the biosensor 200 into the sensor insertion hole 302 of the measuring device 301, and then operating the operation button 303 in accordance with the displayed content of the display unit 304.

Note that the operation button 303 can be used for operations such as various types of setting relating to the measuring device, starting diagnosis, and finishing the diagnosis. The measuring device may include a touch panel that can be used for the above-mentioned operations together with or instead of the operation button.

Further, the display unit 304 is used to display information provided from the analysis output unit of the measuring device 301, setting information relating to diagnosis, a state of progress of measurement, and the like. The display unit 304 may include a liquid crystal panel, a plasma display panel, an electroluminescent panel, or the like. Further, in the case where the display unit includes a touch panel, the display unit may be configured to operate the measuring device, i.e., the operation button and the display unit may be integrally formed.

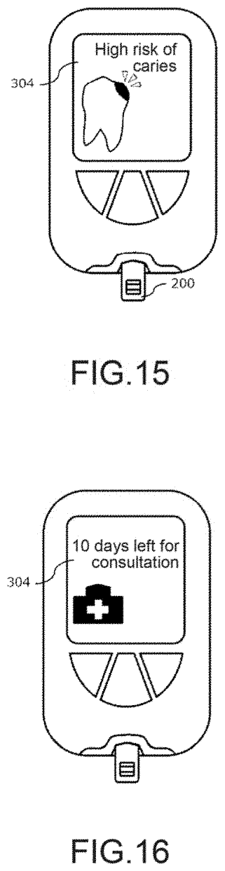

For example, in the case where the current value in the sample solution is greater than or equal to the reference by the above-described measurement flow, “high risk of caries” is displayed on the display unit 304 (FIG. 14). Further, in the case where the current value is greater than or equal to the reference as compared with the previous current value, “with progress of caries” (FIG. 15) is displayed. Further, a recommended consultation deadline to a dental clinic can be displayed, as described below (FIG. 16).

Note that the measuring device 301 includes a measuring unit, a voltage applying unit, a storage unit, an analysis output unit, and a control unit that controls the respective units (which are not shown), and the control unit is a processor. Examples of the control unit include, but not limited to, a central processing unit (CPU), a microprocessor, a processor core, a multiprocessor, an ASIC (application-specific integrated circuit), and an FPGA (field programmable gate array).

Further, the control unit reads the program stored in the storage unit and performs predetermined arithmetic processing in accordance with the program.

Further, the control unit is capable of writing and reading the operation result according to the program to/from the storage unit as appropriate.

Further, the storage function of the storage unit can be realized by a non-volatile memory such as an HDD (hard disk drive) and an SSD (solid state drive). Further, the storage unit may have a function as a memory for writing or reading intermediate progress or the like of the arithmetic processing by the control unit. The memory function of the storage unit can be realized by a volatile memory such as a RAM (Random Access memory) and a DRAM (Dynamic Random Access memory).

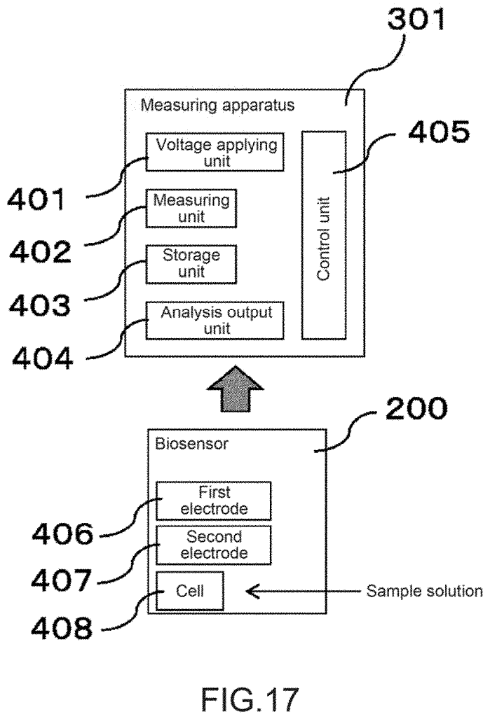

FIG. 17 is a diagram describing a functional configuration of a diagnosing apparatus including the measuring device 301 and the biosensor 200. The measuring device 301 includes a voltage applying unit 401, a measuring unit 402, a storage unit 403, an analysis output unit 404, and a control unit 405 that is a processor controlling the respective units described above.

Further, the biosensor 200 includes a first electrode 406, a second electrode 407, and a cell 408.

First, a sample solution is introduced into the cell 408 so as to come into contact with the first electrode 406 and the second electrode 407. Next, the biosensor 200 and the measuring device 301 are connected to each other, and the first electrode 406 and the second electrode 407 are electrically connected to the voltage applying unit 401 and the measuring unit 402.

Next, the voltage applying unit 401 controlled by the control unit 405 applies a voltage between the first electrode 406 and the second electrode 407, and the measuring unit 402 measures the current value at this time. Next, the analysis output unit 404 controlled by the control unit 405 compares the reference stored in advance in the storage unit 403 with the obtained current value, and outputs information relating to a state of progress of an illness in the oral cavity of a subject, and this is displayed on the display unit that has been described above. Note that the reference is as described above in the description of the diagnosing apparatus according to the first embodiment of the present invention.

In the diagnosing apparatus according to the above-mentioned embodiment, the biosensor is of a cartridge type, and the biosensor may be updated for each sample solution. By doing so, it becomes easier to prevent contamination between different sample solutions on the electrodes.

### (Another Embodiment of Biosensor)

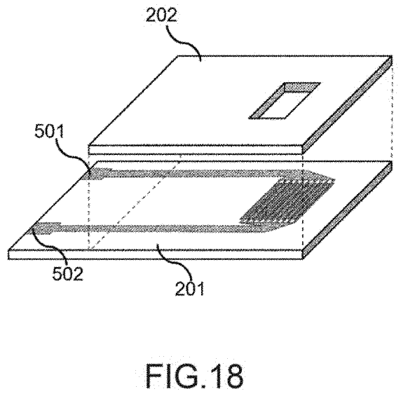

FIG. 18 is a top view showing another embodiment of a biosensor that can be used for the diagnosing apparatus according to the embodiment of the present invention. A biosensor 500 includes a comb-shaped first electrode 501 and a comb-shaped second electrode 502 disposed on the lower insulating substrate 201. A cell is formed by the lower insulating substrate 201 and an opening of the upper insulating substrate 202.

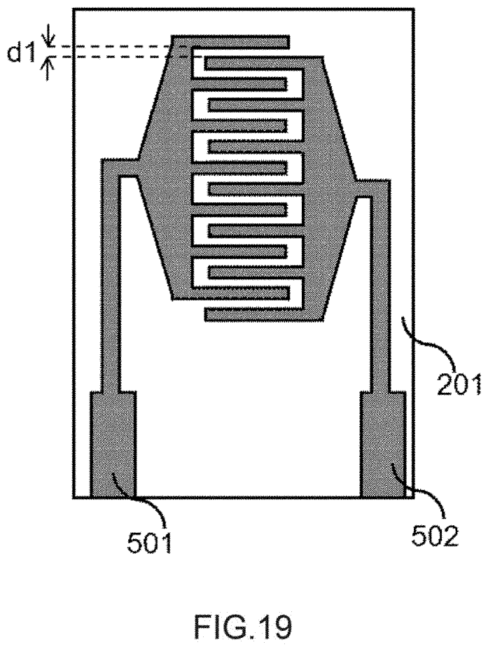

FIG. 19 is a top view schematically showing a state in which the upper insulating substrate 202 of the biosensor 500 has been removed. The comb-shaped first electrode 501 and the comb-shaped second electrode 502 are comb-shaped electrodes corresponding to each other. Note that although both the first electrode and the second electrode are comb-shaped electrodes in the biosensor according to this embodiment, but a response tends to be faster as described above when at least one selected from the group consisting of the first electrode and the second electrode is a comb-shaped electrode.

At this time, the embodiment of the distance dl is as described above.

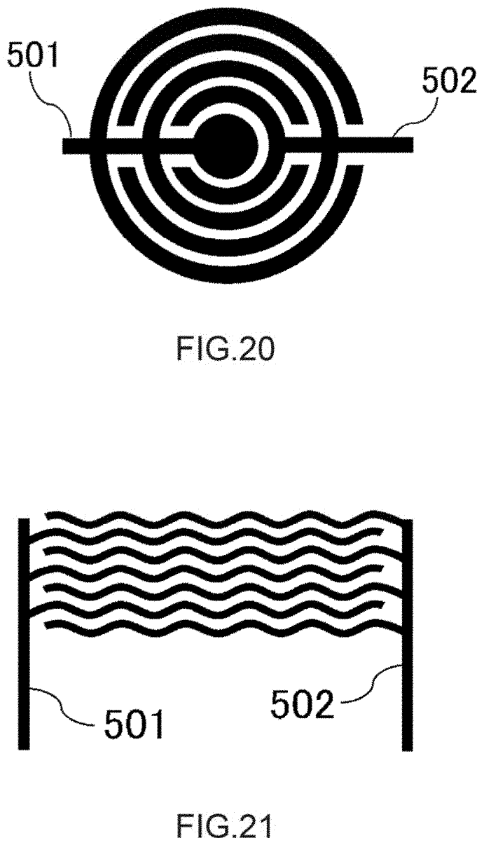

Note that as used herein, a comb-shaped electrode means an electrode configured such that two facing electrodes are combined with each other. Note that the structure of the comb-shaped electrode may be a so-called comb-shape, a concentric shape as shown in FIG. 20, and may be a wave shape as shown in FIG. 21.

### Third Embodiment of Diagnosing Apparatus

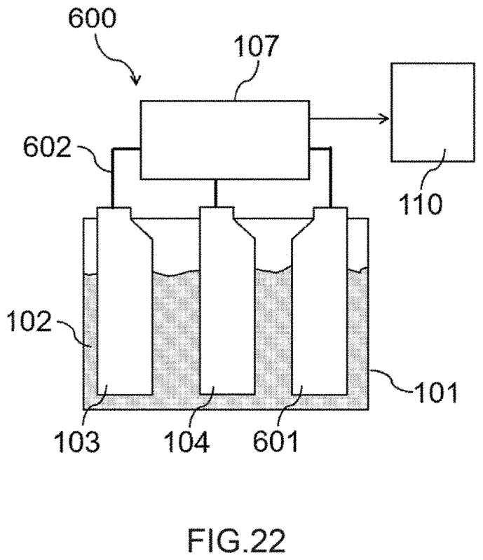

FIG. 22 is a schematic diagram showing a diagnosing apparatus according to a third embodiment of the present invention. A diagnosing apparatus 600 is similar to the diagnosing apparatus according to the first embodiment, which has been described above, except that it further includes a reference electrode 601 in the cell 101 so as to come into contact with the sample solution 102.

Note that the first electrode 103 and the second electrode 104 are electrically connected to the reference electrode 601 via the potentiostat 107.

Note that the diagnosing apparatus 600 includes the first electrode 103, the second electrode 104, and the reference electrode 601, but the diagnosing apparatus according to the embodiment of the present invention is not limited to the above. The diagnosing apparatus may include a counter electrode instead of the reference electrode 601, or may include a first electrode and two reference electrodes, or a first electrode, a second electrode, and two or more reference electrodes.

Note that in the diagnosing apparatus including a reference electrode, the potential of the electrode can be measured, so that a diagnosing apparatus having better effects of the present invention can be achieved.

Note that as the reference electrode, a known reference electrode for an electrochemical measurement cell can be used, and for example, a silver/silver chloride electrode or the like can be used.

Further, as the counter electrode, a known counter electrode for an electrochemical measurement cell can be used.

### Fourth Embodiment of Diagnosing Apparatus

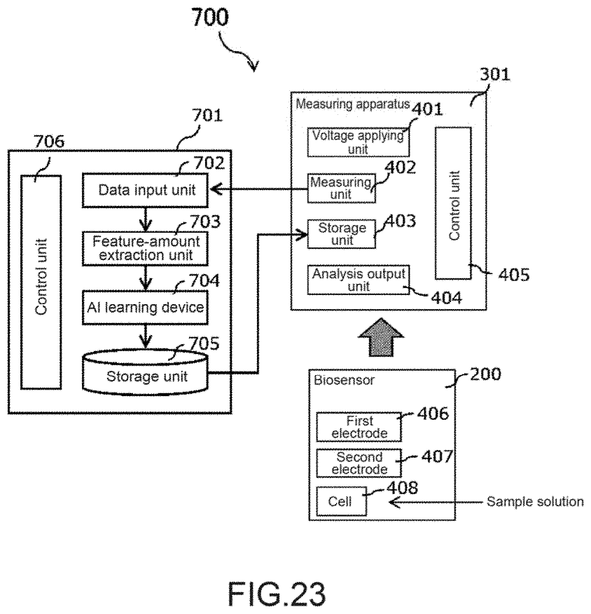

FIG. 23 is a functional explanatory diagram of a diagnosing apparatus 700 including the measuring device 301, the biosensor 200, and a reference creating device 701 that creates a reference by machine-learning on the basis of a response.

The diagnosing apparatus 700 includes the biosensor 200, the measuring device 301, and the reference creating device 701, and the measuring device 301 and the reference creating device 701 are connected so as to be capable of performing data communication with each other.

The reference creating device 701 includes a data input unit 702 that inputs time-varying data of a current value, a feature-amount extraction unit 703 that extracts a feature amount from the input current value, an AI learning apparatus 704 that calculate a reference by machine-learning on the basis of the feature amount, a storage unit 705, and a control unit 706 that is a processor that controls the respective units described above.

The control unit 706 is a processor having a function that controls the respective units of the reference creating device 701. Examples of the control unit 706 include, but not limited to, a central processing unit (CPU), a microprocessor, a processor core, a multiprocessor, an ASIC (application-specific integrated circuit), and an FPGA (field programmable gate array).

Further, the control unit 706 reads the program stored in the storage unit 705 and performs predetermined arithmetic processing in accordance with the program.

Further, the control unit is capable of writing and reading the operation result according to the program to/from the storage unit 705 as appropriate.

Further, the storage function of the storage unit 705 can be realized by a non-volatile memory such as an HDD (hard disk drive) and an SSD (solid state drive). Further, the storage unit may have a function as a memory for writing or reading intermediate progress of the arithmetic processing by the control unit. The memory function of the storage unit can be realized by a volatile memory such as a RAM (Random Access memory) and a DRAM (Dynamic Random Access memory).

Under the control of the control unit 405, the measuring device 301 transmits data (response) relating to the temporal change of the current value relating to a sample solution to the reference creating device 701.

The feature-amount extraction unit 703 extracts information such as an absolute value of the current, a first derivative value of the time, a second derivative value of the time, the time until the rise of the current value, the maximum current value, the time until the maximum current value, and the rising method of the current value from the temporal change (response) of the input current value, and transmits the extracted information (feature amount) to the AI learning apparatus 704.

The AI learning apparatus 704 performs machine-learning on the basis of the feature amount provided from the feature-amount extraction unit 703 and the information relating to the type of an illness and/or a state of progress of the illness. The AI learning apparatus 704 creates a reference defining the relationship between a numerical value obtained from the response as a result of the machine-learning and at least one item selected from the group consisting of the type of and illness and a state of progress of the illness.

The method of machine learning is not particularly limited, and a known method such as a neural network, discriminant analysis, logistic regression analysis, genetic programming, inductive logic programming, support vector machine, and clustering only needs to be used.

Note that the control unit 706 reads the program stored in the storage unit 705 and performs predetermined arithmetic processing in accordance with the program.

Further, the control unit 706 is capable of writing or reading the operation result according to the program to/from the storage unit 705 as appropriate.

Further, the storage function of the storage unit 705 can be realized by a non-volatile memory such as an HDD (hard disk drive) and an SSD (solid state drive). Further, the storage unit 705 may have a function as a memory for writing or reading intermediate progress of the arithmetic processing by the control unit 706. The memory function of the storage unit can be realized by a volatile memory such as a RAM (Random Access memory) and a DRAM (Dynamic Random Access memory).

The reference stored in the storage unit 705 is transmitted to the measuring device 301 by the control unit 706. The measuring device 301 receives the above-mentioned reference, and the received reference is recorded on the storage unit 403 of the measuring device 301 and used for analyses.

### Fifth Embodiment of Diagnosing Apparatus

A diagnosing apparatus according to a fifth embodiment of the present invention is a diagnosing apparatus further including: a transmission unit that transmits a response to an external server via a communication line; and a reception unit that receives at least one item selected from the group consisting of a consultation deadline or expected progress of the illness that has been calculated by machine-learning in an external server, on the basis of the response transmitted from the transmission unit.

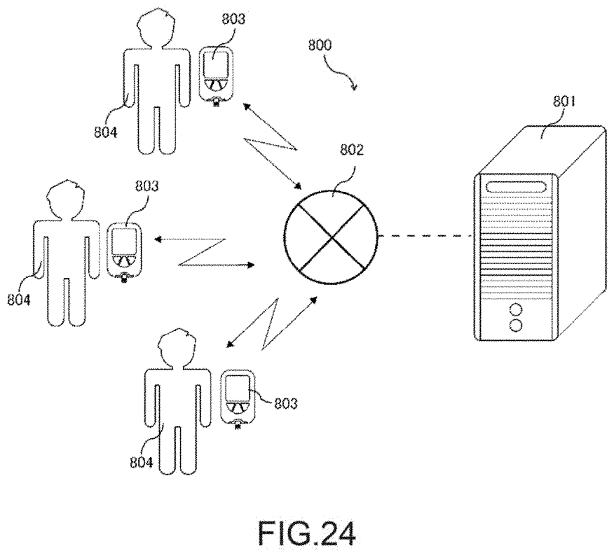

FIG. 24 shows a schematic diagram of a system including a diagnosing apparatus according to the above-mentioned embodiment. A diagnosing apparatus 803 transmits a response (including the feature amount that has been described above) obtained from body fluid of each subject 804 to a server 801 via a network (e.g., the internet) 802. In the server 801, at least one item selected from the group consisting of the consultation deadline or the expected progress of the illness is calculated by the machine-learning that has been described above.

In the above-mentioned server 801, data (response) of others is accumulated, the AI picks up data of a person who shows behavior close to that of the subject, and expectation data relating to the response change for the following days (e.g., increase in the current value) is calculated (expected progress of the illness). Further, also the deadline for visiting the medical clinic (which is defined by how many days left until the reference will be exceeded; the consultation deadline) is calculated.

The diagnosing apparatus 803 receives, from the server 801, at least one item selected from the group consisting of a consultation deadline and expected progress of the illness, and displays it.

### Program (First Embodiment)

A program according to the embodiment of the present invention is a program that causes, in a system that includes the above-mentioned diagnosing apparatus and a server that is a computer, the diagnosing apparatus and the server being configured to communicate with each other via a network, the computer to execute the procedures of; receiving a specific response determined to be abnormal by comparing a numerical value obtained from the response and the reference, of the response obtained by the diagnosing apparatus; applying the specific response to an estimation model to estimate generation of pathogenic microbes in the sample solution, the estimation model being learned to estimate, when an electrochemical response is input, presence or absence of generation of the pathogenic microbes responsible for the illness, learning having been performed in advance in the estimation model by learning data including an electrochemical response of a learning sample solution different from the sample solution and a microbiota analysis result of the learning sample solution; and prompting, where generation of the pathogenic microbes is expected, the display unit to display that microbiota analysis is necessary.

### <System>

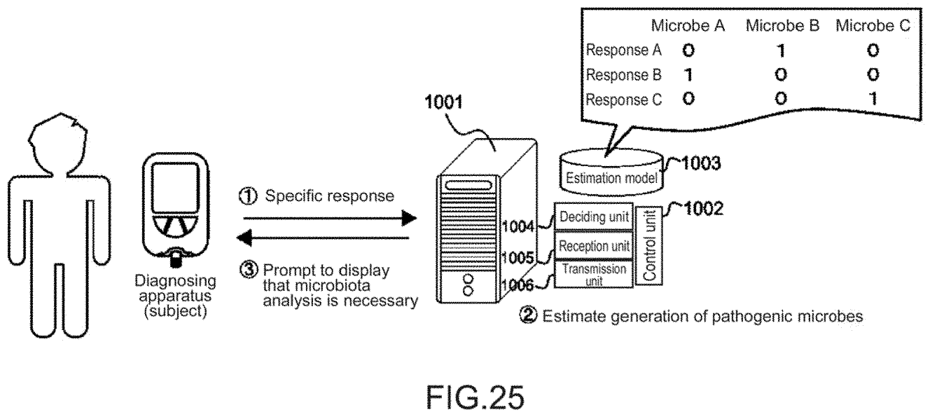

The above-mentioned program is a system including the diagnosing apparatus that has been described above and a server that is a computer, the diagnosing apparatus and the server being configured to be capable of communicating with each other via a network. FIG. 25 shows a schematic diagram showing the system.

In FIG. 25, a server 1001 is a computer that executes a program and performs processing, and specifically includes a control unit 1002, a storage unit 1003, a deciding unit 1004, a reception unit 1005, and a transmission unit 1006.

The control unit 1002 is a processor having a function that controls the respective units of the computer. Examples of the control unit 1002 include, but not limited to, a central processing unit (CPU), a microprocessor, a processor core, a multiprocessor, an ASIC (application-specific integrated circuit), and an FPGA (field programmable gate array).

Further, the control unit 1002 reads the program stored in the storage unit 1003 and performs predetermined arithmetic processing in accordance with the program.

Further, the control unit is capable of writing and reading the operation result according to the program to the storage unit 1003 as appropriate.

Further, the storage function of the storage unit 1003 can be realized by a non-volatile memory such as an HDD (hard disk drive) and an SSD (solid state drive). Further, the storage unit may have a function as a memory for writing or reading intermediate progress of the arithmetic processing by the control unit. The memory function of the storage unit can be realized by a volatile memory such as a RAM (Random Access memory) and a DRAM (Dynamic Random Access memory).

The transmission unit 1006 and the reception unit 1005 transmit/receive various types of data and the like to/from devices connected via a network such as the Internet. Specifically, they may be an I/F that transmits/receives data to/from the diagnosing apparatus by radio or the like.

### <Processing by Server>

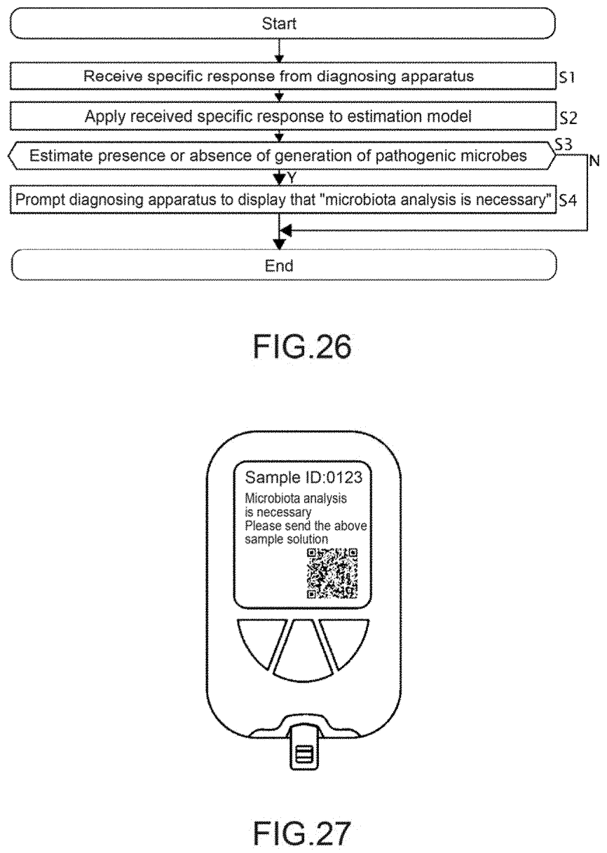

The above-mentioned program is a program executed by a server, and the process flow thereof is shown in FIG. 26.

First, in Step S1, the control unit of the server instructs the reception unit to receive, from the diagnosing apparatus, a specific response determined to be abnormal in the diagnosing apparatus, of an electrochemical response relating to a sample solution, and stores the data in the storage unit. The electrochemical response received from the diagnosing apparatus is as described above. Typical examples thereof include the relationship between the current value and the time of voltage application, and the relationship between the current value and the applied voltage.

Next, in Step S2, the control unit of the server applies the received electrochemical response to the estimation model stored in the storage unit. Learning has been performed in advance in the estimation model by data including electrical application for a learning sample solution and a microbiota analysis result for the above-mentioned learning sample. Because the above-mentioned microbiota analysis result also includes information regarding whether or not the detected microbe corresponds to a pathogenic microbe responsible for an illness, inputting an electrochemical response of the sample solution to the above-mentioned estimation model can estimate the presence or absence of generation of pathogenic microbes (Step S3). In this embodiment, learning is performed in accordance with a learning model constructed by a neural network including a multi-layer neural network. The learning model constructed by the neural network including an input-layer, an output-layer, and an intermediate layer is capable of using any suitable method. For example, a CNN (Convolutional Neural Network) can also be applied.

Note that the above-mentioned Step S2 and Step S3 are executed by the control unit controlling the deciding unit and calling the estimation model stored in the storage unit.

Next, in the above-mentioned Step S3, in the case where the generation of pathogenic microbes is expected in the sample solution, the control unit controls the transmission unit to prompt the display unit of the diagnosing apparatus to display that microflora analysis is necessary for the corresponding sample solution.

FIG. 27 shows an example of displaying that microflora analysis is necessary. In FIG. 27, a sample solution for which microflora analysis is necessary is identified by the sample ID assigned each time of measurement, and an indication that microbiota analysis is necessary for the sample solution relating to the sample ID and a two-dimensional barcode (e.g., “QR Code (registered trademark)”) in which the procedure of the microbiota analysis has been stored are displayed. Examples of the procedure of microbiota analysis include a method for transmitting a sample solution for microbiota analysis to an analysis institution having an analysis device, and a deadline for transmission.

### Program (Second Embodiment)

A program according to the embodiment of the present invention is a program that causes, in a system that includes the above-mentioned diagnosing apparatus, a server that is a computer, and an analysis device capable of performing microbiota analysis, the diagnosing apparatus, the server, and the analysis device being configured to communicate with each other via a network, the computer to execute the procedures of; receiving a specific response determined to be abnormal by comparing a numerical value obtained from the response and the reference, of the response obtained by the diagnosing apparatus, and a sample ID corresponding to the sample solution; applying the specific response to an estimation model to estimate generation of pathogenic microbes in the sample solution, the estimation model being learned to estimate, when an electrochemical response is input, presence or absence of generation of the pathogenic microbes responsible for the illness, learning having been performed in advance in the estimation model by learning data including an electrochemical response of a learning sample solution different from the sample solution and a microbiota analysis result of the learning sample solution; prompting, where generation of the pathogenic microbes is expected, the display unit to display that microbiota analysis is necessary for the sample solution; receiving a result of microbiota analysis of the measurement solution and the sample ID from the analysis device; deciding presence or absence of pathogenic microbes in the sample solution from the result of microbiota analysis; and prompting, where it is decided from the result of microbiota analysis that the pathogenic microbes are not generated, the diagnosing apparatus to change the reference.

### <System>

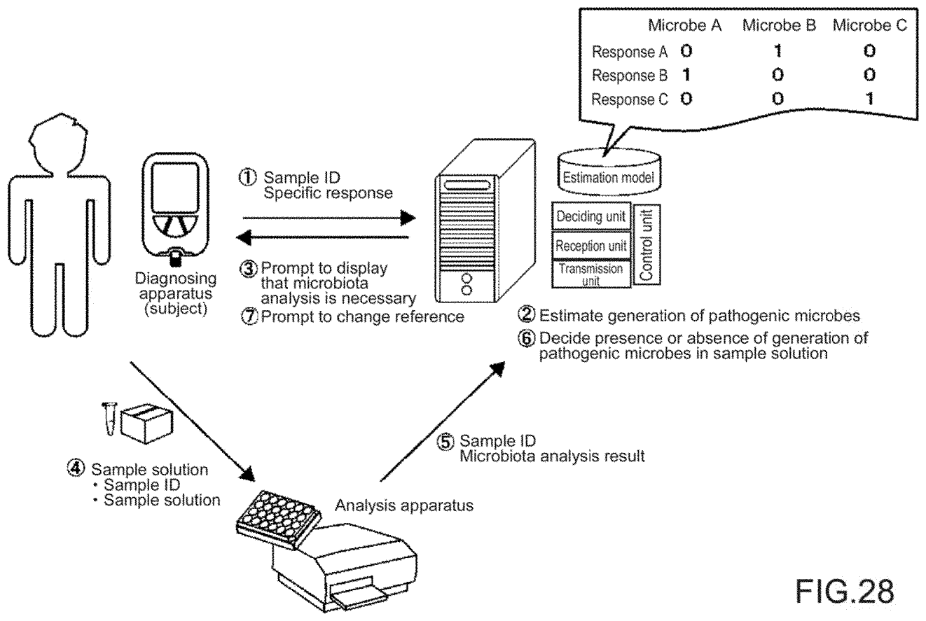

The above-mentioned program is a system including the diagnosing apparatus that has been described above, a server that is a computer, and an analysis device capable of performing microbiota analysis, the diagnosing apparatus, the server, and the analysis device being configured to be capable of communicating with each other via a network. FIG. 28 shows a schematic diagram of the system.

In FIG. 28, a diagnosing apparatus and a server are similar to the diagnosing apparatus and the server in the system to which the program according to the first embodiment that has been described above is applied.

### <Processing by Server>

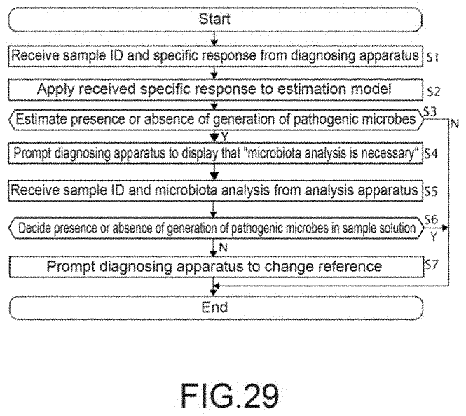

The above-mentioned program is a program executed by a server, and the process flow thereof is shown in FIG. 29.

First, in Step S1, the control unit of the server instructs the reception unit to receive, from the diagnosing apparatus, a specific response determined to be abnormal in the diagnosing apparatus, of an electrochemical response relating to a sample solution, and stores the data in the storage unit. The electrochemical response received from the diagnosing apparatus is as described above. Typical examples thereof include the relationship between the current value and the time of voltage, and the relationship between the current value and the applied voltage.

Next, in Step S2, the control unit of the server applies the received electrochemical response to the estimation model stored in the storage unit, which has been described above. Inputting an electrochemical response of a sample solution to the above-mentioned estimation model can estimate the presence or absence of generation of pathogenic microbes (Step S3).

Next, in the case where the generation of pathogenic microbes is expected in a sample solution in the above Step S3, the control unit controls the transmission unit to output, on the display unit of the diagnosing apparatus, an indication that microflora analysis is necessary for the corresponding sample solution (Step S4).

Next, in Step S5, the control unit of the server controls the reception unit to receive, from the analysis device, the sample ID and the microbiota analysis result relating to the sample solution, and stores them in the storage unit. This sample ID is a sample solution commonly assigned to the sample solution whose response has been measured in the diagnosing apparatus and a sample solution for which microbiota analysis has been performed in the analysis device, and is one allocated by the control unit of the diagnosing apparatus as a value unique to the sample solution.

Next, in Step S6, the control unit of the server controls the deciding unit to determine the generation of the presence or absence of pathogenic microbes in the sample solution from the microbiota analysis result.

As a result, in the case where it is determined that pathogenic microbes have not been generated, the control unit controls the deciding unit to create a new reference that is determined to be normal for the above-mentioned specific response, and instructs the transmission unit to transmit the new reference and prompt the diagnosing apparatus to change the reference (Step S7).

At this time, the new reference does not necessarily need to be transmitted, and the control unit only needs to prompt the transmission unit to change the reference. In this case, a new reference is created in the diagnosing apparatus.

In accordance with the above-mentioned program, it is possible to update, in the case where a pathogenic microbe is not detected by microbiota analysis (false positive) in a response determined to be abnormal in the diagnosing apparatus, the reference of the diagnosing apparatus and cause the diagnosing apparatus to provide more accurate information.

### REFERENCE SIGNS LIST

100: diagnosing apparatus

101: cell

102: sample solution

103: first electrode

104: second electrode

105: voltage applying unit

106: measuring unit

107: potentiostat

108: storage unit

109: analysis output unit

110: terminal

111: circuit

200: biosensor

201: lower insulating substrate

202: upper insulating substrate

203: pipette

300: diagnosing apparatus

301: measuring device

302: sensor insertion hole

303: operation button

304: display unit

401: voltage applying unit

402: measuring unit

403: storage unit

404: analysis output unit

405: control unit

406: first electrode

407: second electrode

408: cell

500: biosensor

501: first electrode

502: second electrode

600: diagnosing apparatus

601: reference electrode

700: diagnosing apparatus

701: reference creating device

702: data input unit

703: feature-amount extraction unit

704: AI learning apparatus

705: storage unit

706: control unit

801: server

803: diagnosing apparatus

804: subject

901: measuring device

1001: server

1002: control unit

1003: storage unit

1004: deciding unit

1. A diagnosing apparatus, comprising:
a cell into which a sample solution containing body fluid collected from a subject is introduced;
at least two electrodes that are disposed in the cell and come into contact with the sample solution;
a voltage applying unit that applies a voltage between the electrodes;
a measuring unit that measures an electrochemical response when the voltage is applied;
a storage unit that stores a reference that defines a relationship between a numerical value obtained from the response and at least one of a type of an illness or a state of progress of the illness; and
an analysis output unit that compares the numerical value obtained from the subject with the reference, and provides information for determining at least one of the type of the illness that has developed in the subject or the state of progress of the illness that has developed in the subject.

2. The diagnosing apparatus according to claim 1, wherein
the numerical value is an absolute value of a current measured by the measuring unit or a difference between the response measured using body fluid obtained from the subject and the response measured using body fluid obtained from at least one of the subject in a healthy state or a healthy person different from the subject.

3. The diagnosing apparatus according to claim 1, wherein the body fluid is saliva.

4. The diagnosing apparatus according to claim 3, wherein the illness is at least one of caries or a periodontal disease.

5. The diagnosing apparatus according to claim 1, wherein the electrodes include at least one of a reference electrode or a counter electrode.

6. The diagnosing apparatus according to claim 1, wherein the voltage applying unit applies a predetermined voltage between the electrodes.

7. The diagnosing apparatus according to claim 1, wherein the voltage applying unit applies a sweep voltage between the electrodes.

8. The diagnosing apparatus according to claim 1, wherein the storage unit records the measured response, and
the analysis output unit compares the response obtained by measurement of the sample solution with the response that has been obtained by preceding measurement prior to the measurement and recorded in the storage unit, and provides information for determining the at least one of the type of the illness that has developed in the subject or the state of progress of the illness on a basis of the reference from a result of the comparison.

9. The diagnosing apparatus according to claim 1, wherein the cell is configured to be hermetically sealable.

10. The diagnosing apparatus according to claim 1, wherein a shortest distance between the electrodes in the cell is a distance at which one of cells of a microbe responsible for the illness can be short-circuited.

11. The diagnosing apparatus according to claim 1, wherein the shortest distance between the electrodes in the cell is 700 nm to 2,000 nm.

12. The diagnosing apparatus according to claim 1, wherein the electrode includes a comb-shaped electrode.

13. The diagnosing apparatus according to claim 1, further comprising:
a transmission unit that transmits the response to an external server via a communication line; and
a reception unit that receives at least one of a consultation deadline or expected progress of the illness that has been calculated by machine-learning in an external server on a basis of the response transmitted from the transmission unit.

14. The diagnosing apparatus according to claim 1, further comprising
a reference creating device that create the reference by machine-learning on a basis of the above response.

15. An analysis method, comprising:
a step of introducing, into a cell in which at least two electrodes are disposed, a sample solution containing body fluid collected from a subject so as to come into contact with the electrodes;
a step of measuring an electrochemical response when a voltage is applied between the electrodes; and
a step of comparing a reference defining a relationship between a numerical value obtained from the response and at least one of a type of an illness or a state of progress of the illness with the numerical value obtained from the subject to provide information for determining at least one of the type of the illness or the state of progress of the illness in the subject.

16. The analysis method according to claim 15, wherein the applied voltage is a predetermined voltage.

17. The analysis method according to claim 15, wherein the applied voltage is a sweep voltage.

18. The analysis method according to claim 15, wherein the body fluid is saliva.

19. The analysis method according to claim 18, wherein the illness is at least one of caries or a periodontal disease.

20. A program that causes, in a system that includes a diagnosing apparatus and a server that is a computer, the diagnosing apparatus and the server being configured to communicate with each other via a network, the diagnosing apparatus including
a cell into which a sample solution containing body fluid collected from a subject is introduced,
at least two electrodes that are disposed in the cell and come into contact with the sample solution,
a voltage applying unit that applies a voltage between the electrodes,
a measuring unit that measures an electrochemical response when the voltage is applied,
a storage unit that stores a reference that defines a relationship between a numerical value obtained from the response and at least one of a type of an illness or a state of progress of the illness,
an analysis output unit that compares the numerical value obtained from the subject with the reference, and provides information for determining at least one of the type of the illness that has developed in the subject or the state of progress of the illness that has developed in the subject, and
a display unit,
the computer to execute the procedures of;
receiving a specific response determined to be abnormal by comparing the numerical value and the reference, of the response obtained by the diagnosing apparatus;
applying the specific response to an estimation model to estimate generation of pathogenic microbes in the sample solution, the estimation model being learned to estimate, when an electrochemical response is input, presence or absence of generation of the pathogenic microbes responsible for the illness, learning having been performed in advance in the estimation model by learning data including an electrochemical response of a learning sample solution different from the sample solution and a microbiota analysis result of the learning sample solution; and
prompting, where generation of the pathogenic microbes is expected, the display unit to display that microbiota analysis is necessary.

21. A program that causes, in a system that includes a diagnosing apparatus, a server that is a computer, and an analysis device capable of performing microbiota analysis, the diagnosing apparatus, the server, and the analysis device being configured to communicate with each other via a network,
the diagnosing apparatus including
a cell into which a sample solution containing body fluid collected from a subject is introduced, at least two electrodes that are disposed in the cell and come into contact with the sample solution,
a voltage applying unit that applies a voltage between the electrodes,
a measuring unit that measures an electrochemical response when the voltage is applied,
a storage unit that stores a reference that defines a relationship between a numerical value obtained from the response and at least one of a type of an illness or a state of progress of the illness,
an analysis output unit that compares the numerical value obtained from the subject with the reference, and provides information for determining at least one of the type of the illness that has developed in the subject or the state of progress of the illness that has developed in the subject, and
a display unit,
the computer to execute the procedures of;
receiving a specific response determined to be abnormal by comparing the numerical value and the reference, of the response obtained by the diagnosing apparatus, and a sample ID corresponding to the sample solution;
applying the specific response to an estimation model to estimate generation of pathogenic microbes in the sample solution, the estimation model being learned to estimate, when an electrochemical response is input, presence or absence of generation of the pathogenic microbes responsible for the illness, learning having been performed in advance in the estimation model by learning data including an electrochemical response of a learning sample solution different from the sample solution and a microbiota analysis result of the learning sample solution;
prompting, where generation of the pathogenic microbes is expected, the display unit to display that microbiota analysis is necessary for the sample solution;
receiving a result of microbiota analysis of the measurement solution and the sample ID from the analysis device;
deciding presence or absence of pathogenic microbes in the sample solution from the result of microbiota analysis; and
prompting, where it is decided from the result of microbiota analysis that the pathogenic microbes are not generated, the diagnosing apparatus to change the reference.
